(19) United States
(12) Patent Application Publication
(10) Pub. No.: US 2017/0043150 A1
(43) Pub. Date: Feb. 16, 2017

(54) SKIN CARE DEVICE

(71) Applicant: Seoulin Medi Care Co., Ltd., Seongnam-Si (KR)

(72) Inventor: Byoung-Choul KIM, Seongnam-Si (KR)

(73) Assignee: Seoulin Medi Care Co., Ltd., Seongnam-Si (KR)

(21) Appl. No.: 14/825,530

(22) Filed: Aug. 13, 2015

(51) Int. Cl. A61M 37/00

(52) U.S. Cl. CPC A61M 37/0092; A61M 2037/0007

(57) ABSTRACT

A skin care device is provided. The skin care device includes a hand piece and a tip which is mounted at a front end of the hand piece and is configured to peel impurities off the surface of a skin as the tip contacts the skin. In the center of a front end surface of the tip, there is provided an input hole through which face wash liquid is inputted toward a skin, and at an edge portion thereof, there is formed a suction hole for sucking and discharging the face wash liquid, and a groove-shaped flow passage for guiding the flow of the face wash liquid between the input hole and the suction hole is formed at a front end surface of the tip, and a vibrator is mounted at an inner side of the tip, thus concurrently supplying vibrations to the tip and the face wash liquid.

### TECHNICAL FIELD

The present invention relates to a skin care device, and in particular to a skin care device which allows to effectively remove keratin, sebum, etc. from a face skin while efficiently penetrating nutrition components contained in face wash liquid into a skin.

### BACKGROUND ART

A skin care device is directed to a device configured to remove keratin from a face and to penetrate a liquid containing skin recovery nutrition into a skin. Such a skin care device is disclosed in the Korean patent publication number 10-2010-0039597 (a tip for a skin care device) and the US patent publication number US2007/0156124 (apparatus and methods for treating the skin), of which the Korean patent publication number 10-2010-0039597 will be described as a relevant art.

As for the configuration of the tip for a conventional skin care device, the tip 1 for a skin care device is mounted at a front end of a hand piece 2 forming the skin care device and contacts with a skin and then peels impurities (keratin, sebum, etc.) off the surface of a skin and includes a ring-shaped peeling-off shoulder 3 which is configured to peel impurities off the front surface. The conventional tip for a skin care device further includes an input hole 4 formed on an inner circumferential surface of the peeling-off shoulder 3 so as to receive a face wash liquid “W”, and a suction hole 5 for discharging the face wash liquid to the outside. In this configuration, the input hole 4 and the suction hole 5 of the tip 1 are formed at an inner side of the peeling-off shoulder 3 and extends in an outward direction and communicate with a spiral groove 7 formed of assistant peeling-off shoulders 6 provided in a vortex shape.

When in use, the above tip 1 for a skin care device is attached detachable on a front end of the skin care device.

With the tip 1 being mounted at a front end of the hand piece 2, when it is pushed while surface-contacting with a users skin, the peeling-off shoulder 3 and the assistant peeling-off shoulder 6 come into contact with the skin, so impurities, for example, keratin, sebum, etc. on the surface of the skin can be peeled off.

At this time, the face wash liquid “W” inputted from the skin care device through the input hole 4 and the hand piece 2 is inputted into the inside of the peeling-off shoulder 3, and the inputted face wash liquid “W” discharges to the outside through the spiral groove 7 and the suction hole 5.

In the above-described operations, functional components contained in the face wash liquid “W” penetrate into the side of the skin, thus activating the skin, whereupon the health of the skin can be improved, and the impurities (keratin, sebum, etc.) which peel off in the passage of the face wash liquid “W” which discharge into the suction hole 5 through the spiral hole 7 are collected and discharged to the outside.

Namely, the efficiency of penetration of functional components into the skin can be enhanced by improving the contact time and efficiency with respect to the skin in such a way to prolong the flow time and distance of the face wash liquid “W”, and discharge efficiency based on contact time with impurities and the increase of surface area can be improved.

The conventional skin care device can remove keratin and sebum only by a face wash liquid “W”, however if keratin, sebum, etc. are adhered on a skin or if sebum, etc. are in hair follicles, it is not easy to peel off, whereupon the effects of skin cares are low.

Since the nutrition components contained in the face wash liquid “W” just contact with the surface of the skin, they cannot penetrate deep into the skin.

Since a lot of impurities are mixed in the face wash liquid “W”, impurities may easily adhere in the passage which is formed through the spiral groove 7, whereupon the spiral groove 7 may be frequently blocked, and the use of the skin care device may be interrupted.

### DISCLOSURE OF THE INVENTION

Accordingly, it is an object of the present invention to provide a skin care device wherein impurities, for example, keratin, sebum, etc. can be effectively removed by vibrations which generate by a vibrator mounted at a portion of a tip for a hand piece type beauty device, and nutrition components of a face wash liquid can efficiently penetrate deep into a skin, thus maximizing the effects of skin care.

It is another object of the present invention to provide a skin care device wherein a passage formed between an input hole into which face wash liquid is inputted and a suction hole for sucking face wash liquid is divided into multiple parts, thus preventing the passage from being blocked.

To achieve the above objects, there is provided a skin care device, which may include, but is not limited to, a hand piece; and a tip which is mounted at a front end of the hand piece and is configured to peel impurities off the surface of a skin as the tip contacts with the skin, wherein in the center of a front end surface of the tip, there is provided an input hole through which face wash liquid is inputted toward a skin, and at an edge portion thereof, there is formed a suction hole for sucking and discharging the face wash liquid, and a groove-shaped flow passage for guiding the flow of the face wash liquid between the input hole and the suction hole is formed at a front end surface of the tip, and a vibrator is mounted at an inner side of the tip, thus concurrently supplying vibrations to the tip and the face wash liquid.

The flow passage may include, but is not limited to, a first wall part which protrudes from an outer side of an edge of the input hole while surrounding the input hole and includes an open part in such a way that both opposing sides are open; a second wall part which protrudes at a predetermined interval surrounding apart from an outer side of the first wall part and includes an open part at a portion which is different from the open part of the first wall part; and a third wall part which protrudes at a predetermined interval surrounding apart from an outer side of the second wall part and includes an open part at a portion which is different from the open part of the second wall part.

### INDUSTRIAL EFFECTS

According to the present invention, impurities, for example, skin keratin or sebum can be effectively removed in such a way to crush the particles of face wash liquid with the aid of vibrations by generating ultrasonic wave or magnetic vibrations at a portion of a tip of the device, and at the same time the particles of nutrition components contained in the face wash liquid can be crushed into minute pieces, so such nutrition components can penetrate deep into the skin with the aid of vibrations, thus maximizing the effect of skin care.

In addition, the flow passage of the face wash liquid is divided into multiple parts. Even though one of the flow passage parts is blocked, the flow of the face wash liquid is not interrupted, thus obtaining a non-stop skin care operation.

### BRIEF DESCRIPTION OF THE DRAWINGS

The present invention will become better understood with reference to the accompanying drawings which are given only by way of illustration and thus are not limitative of the present invention, wherein;

FIG. 1 is a view illustrating a front side separation structure of a skin care device according to the present invention;

FIG. 2 is a view illustrating a backside separation structure of a skin care device according to the present invention; and

FIG. 3 is a cross sectional view illustrating an assembled state according to the present invention.

### MODES FOR CARRYING OUT THE INVENTION

The present invention will be described with reference to the accompanying drawings.

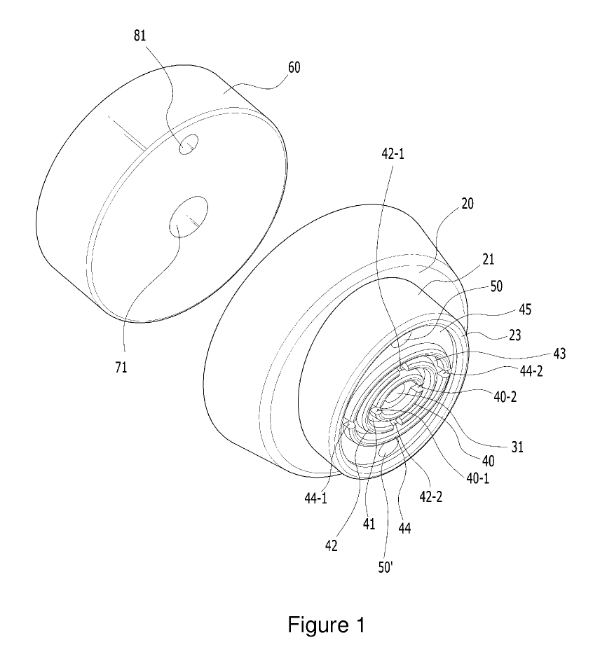

FIG. 1 is a view illustrating a separated portion when viewing a structure of a portion of a tip of a skin care device according to the present invention, wherein a tip 20 includes an elliptical protrusion part 21 formed at a front side thereof, a front end of the protrusion part 21 being inclined in one direction.

An outer edge part 23 protrudes from a front end portion of the protrusion part 21 and along an outer edge portion thereof. An input hole 31 is formed in the center of a front end portion of the protrusion part 21 so as to input face wash liquid into a skin. At an edge portion thereof, there are provided a pair of suction holes 50 and 50′ for sucking face wash liquid wherein the suction holes 50 and 50′ are opposite to each other.

On the front end surface of the protrusion part 21, a flow passage through which face wash liquid flows is formed between the input hole 310 and the suction holes 50 and 50′. The flow passage is formed in such a way that a first wall part 40 protrudes toward a front surface portion along an outer circumferential portion of the input hole 31 and surrounds it. Open parts 40-1 and 40-2 are formed in such a way that the left and right sides of the first wall part 40 are open.

A second wall part 42 formed at a predetermined interval apart outward from the first wall part 40 is formed protruding and surrounding the first wall part 40. Open parts 42-1 and 42-2 are formed in upward and downward directions at deviated at an angle of 90° with respect to the open parts 40-1 and 40-2.

In addition, a third wall part 44 formed at a predetermined interval apart outward from the second wall part 42 is formed protruding and surrounding the second wall part 42. Open parts 44-1 and 44-2 are formed in left and right directions deviated at an angle of 90° with respect to the open parts 42-1 and 42-2.

The heights of the first wall part 40, the second wall part 42 and the third wall part 44 are determined same as the height of an outer part 23. The bottoms of the open parts 40-1 and 40-2, 42-1 and 42-2 and 44-1 and 44-2 have the same heights as the bottom of the front end surface of the protrusion part 21 and are formed flat.

A first flow passage 41 is formed between the first wall part 40 and the second wall part 42 and by the wall parts 40, 42 and 44. A second flow passage 43 is formed between the second wall part 42 and the third wall part 44, and a third flow passage 45 is formed between the third wall part 44 and the outer part 23.

An inner insertion part 60 is formed in a circular plate shape and is inserted from the rear side of the tip 20 to the inner side. In the center of the inner insertion part 60, a through hole 71 is formed. At a portion corresponding to the suction hole 50, a through hole 81 is formed.

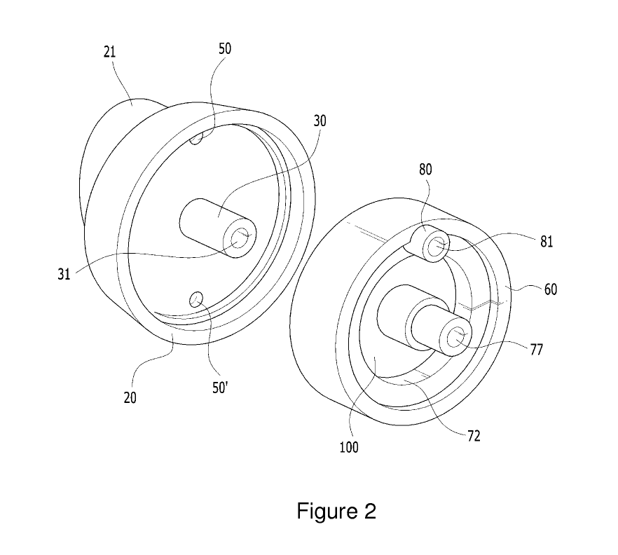

FIG. 2 is a view illustrating a backside configuration of the tip 20 and the inner insertion part 60. At a rear side portion of the tip 20, there is formed a mounting space 22 into which the inner insertion part 60 is inserted fixed. A circular pillar shaped insertion part 30 is formed there, which is installed inserted from the central portion of the engaging space 22, and an input hole 31 is formed passing through the insertion part 30.

In addition, a shoulder part 21 is formed along an edge portion of the engaging space 22, and the suction holes 50 and 50′ are formed at both opposing outer sides.

An input flow passage 70 formed in a circular pillar shape is formed in the center of the backside of the inner insertion part 60. A through hole 71 is formed passing through the input flow passage 70. At a portion corresponding to the suction hole 50, a suction flow passage 80 formed in a circular pillar shape is formed, and a through hole 81 passes through the suction flow passage 80.

An engaging groove 72 is formed along a surrounding portion of the input flow passage 70. A ring shaped vibrator 100 is fixedly inserted into the engaging groove 72.

The vibrator 100 may be formed of an ultrasonic wave vibrator having a vibration frequency of 30 KHz to 1 MHz or a magnetic vibrator having 300 Hz to 800 Hz.

The tip 20 is designed to vibrate by the vibrations from the vibrator 100, and face wash liquid which is inputted through the input hole 31 can be crushed into minute particles.

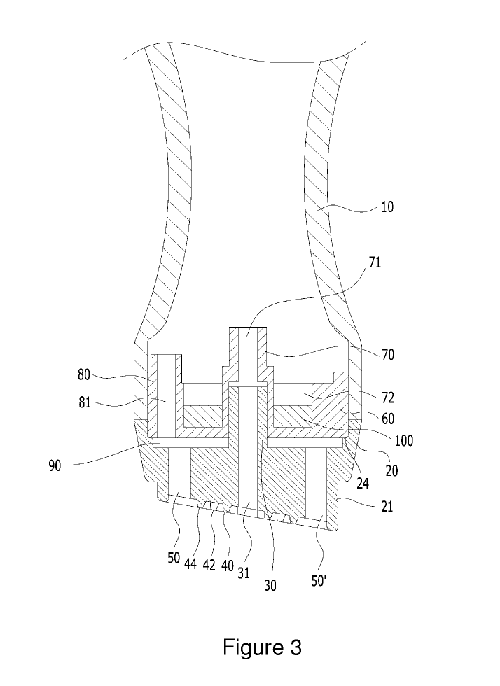

As illustrated in FIG. 3, the inner insertion part 60 is inserted into the inner insertion part 60 of the engaging space 22 of the tip 20. At this time, the insertion part 30 is inserted into the inner side of the input flow passage 70, and the insertion hole 31 and the through hole 71 communicate with each other, and the front portion of the inner insertion part 60 is hooked by the shoulder part 24, thus forming a space part 90 between the backside of the tip 20 and the front surface of the inner insertion part 60.

When the inner insertion part 60 is engaged to the tip 20, the tip 20 can be engaged to the hand piece 10.

The operations of the present invention will be described. With an end portion of the tip 20 contacting with a skin, face wash liquid containing nutrition components is inputted through the through hole 71 and the input hole 31, the face wash liquid flows through the first flow passage 41, the second flow passage 43 and the third flow passage 45 with the aid of the open parts 40-1 and 40-2, 42-1 and 42-2 and 44-1 and 44-2 by the suction force generated through the suction flow passage 80, whereupon impurities, for example, skin keratin, sebum, etc. can be removed.

If any of the flow passages 41, 43 and 45 is blocked, the face wash liquid may detour with the aid of the open parts 40-1 and 40-2, 42-1 and 42-2 and 44-1 and 44-2 formed at both sides of each of the wall parts 40, 42 and 44, so flows take place toward the suction holes 50 and 50′.

The ultrasonic wave vibrations generated by the vibrator 100 transfer to the tip 20 through the inner insertion part 60, whereupon the tip 20 vibrates, thus efficiently peeling off keratin and sebum, and since the ultrasonic wave vibrations transfer to hair follicles, thus efficiently removing sebum from the hair follicles.

Since the ultrasonic wave vibrations generated by the vibrato 100 transfer to the input flow passage 70, the particles of face wash liquid can be crushed into minute particles, so the particles of nutrition components can be crushed into minute particles, which will penetrate into the skin through the flow passages 41, 43 and 45.

In this case, the penetration into skin can be efficient thanks to the particles which have been crushed into minute particles, and the nutrition components can be efficiently inputted into the skin with the aid of ultrasonic wave vibrations.

The face wash liquid which has passed through the flow passages 41, 43 and 45 contains a lot of impurities, for example, keratin, sebum, etc, and such face wash liquid is collected in the space part 90 through the suction holes 50 and 50′ and is sucked into the through hole 81 of the suction flow passage 80 and is discharged to the outside.

Meanwhile, in case where the magnetic vibrator of 300 Hz to 3000 Hz is used, since stronger vibrations can apply to the skin than the ultrasonic wave vibrations, the input of the nutrition as well as skin massage effects can be obtained.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

What is claimed is:

1. A skin care device, comprising:
a hand piece; and
a tip which is mounted at a front end of the hand piece and is configured to peel impurities off the surface of a skin as the tip contacts with the skin, wherein in the center of a front end surface of the tip, there is provided an input hole through which face wash liquid is inputted toward a skin, and at an edge portion thereof, there is formed a suction hole for sucking and discharging the face wash liquid, and a groove-shaped flow passage for guiding the flow of the face wash liquid between the input hole and the suction hole is formed at a front end surface of the tip, and a vibrator is mounted at an inner side of the tip, thus concurrently supplying vibrations to the tip and the face wash liquid.

2. The device of claim 1, wherein the flow passage comprises:
a first wall part which protrudes from an outer side of an edge of the input hole while surrounding the input hole and includes an open part in such a way that both opposing sides are open;
a second wall part which protrudes at a predetermined interval surrounding apart from an outer side of the first wall part and includes an open part at a portion which is different from the open part of the first wall part; and
a third wall part which protrudes at a predetermined interval surrounding apart from an outer side of the second wall part and includes an open part at a portion which is different from the open part of the second wall part.

3. The device of claim 1, wherein the tip comprises:
an insertion part which is inserted into an inner side of the central portion thereof, the input hole being formed passing through the insertion part;
an inner insertion part which is sized to be mounted at an inner side of the tip, wherein an input flow passage is inserted into an inner side of the central portion and includes a through hole and is configured to input the face wash liquid while fixing by inserting the insertion part into an inner side of the through hole, and there is provided a suction flow passage at a portion corresponding to the suction hole at an edge portion thereof; and
a ring-shaped vibrator which is engaged inserted into an outer side of the input flow passage.

4. The device of claim 1, wherein the vibrator is either an ultrasonic wave vibrator or a magnetic vibrator.
